(19) United States
(12) Patent Application Publication
(10) Pub. No.: US 2003/0128034 A1
(43) Pub. Date: Jul. 10, 2003

(54) Medical examination apparatus with optically enlarged interior

(76) Inventor: Hans-Juergen Haumann, Erlangen (DE)

Correspondence Address: SCHIFF HARDIN & WAITE, 6600 SEARS TOWER, 233 S WACKER DR, CHICAGO, IL, 60606-6473, US

(21) Appl. No.: 10/306,520

(22) Filed: Nov. 27, 2002

(30) Foreign Application Priority Data
Nov. 28, 2001 (DE) 10158313.3

(51) Int. Cl. G01V 3/00; A61B 5/05

(52) U.S. Cl. 324/318; 600/411; 600/427

(57) ABSTRACT

A medical examination apparatus, particularly an MR scanner, has an essentially tubular, tight examination space, and a stereoscopic image carrier, particularly metal-free, that simulates a larger space is arranged in the examination space in the field of view of the patient.

### BACKGROUND OF THE INVENTION

1. Field of the Invention

The invention is directed to a medical examination apparatus, particularly an MR scanner, of the type having an essentially tubular, tight examination space.

2. Description of the Prior Art

Due to the relatively tight examination space in devices of the above type, MR examinations cause discomfort to many patients due to a feeling of constriction, up to claustrophobia, particularly given examinations of the head. An actual enlargement of the examination space, however, is not possible or is possible only to a limited extent with great difficulty.

In order to combat this claustrophobic feeling, it has been proposed to provide mirrors above the head of the patient with which the patient can look toward the outside, or to provide an item referred to as “3D goggles” with miniaturized displays. In both instances, the patient no longer sees only the inside wall of the tubular examination space, but these aids still do not convey a true feeling of adequate openness.

### SUMMARY OF THE INVENTION

An object of the present invention is to provide a medical examination apparatus of the type initially described wherein a significantly larger interior can be simulated for the patient in a realistic way, so that claustrophobic anxieties will no longer occur so easily.

This object is achieved in accordance with the invention in a medical examination apparatus having a substantially tubular, tight examination space, and having a stereoscopic image carrier, particularly metal-free, that simulates a larger space, arranged in the field of view of the patient. With the assistance of such stereoscopic methods, a much larger examination space can be simulated for the patient. This solution, moreover, is possible without employing metallic materials, so a utilization in an MR examination space is possible. The stereoscopic images can be composed of plastic, with illumination, if needed, preferably ensuing with light-guiding fibers that are connected to a light source arranged outside the examination space.

In the simplest case, the stereoscopic image carrier can be formed by two sub-images introduced into stereoscopic eyeglasses.

The image carrier preferably is fashioned as a stereoscopic film. This has the advantage that the image carrier can be arced in the shape of a cylindrical jacket, so that it can cover the entire field of vision of the patient with a constant spacing from the patient's face. In addition to the use of stereoscopic eyeglasses that has already been addressed, all other stereoscopic methods can be utilized in the inventive optical enlargement of the examination space of, a medical examination apparatus.

Thus, for example, the image can be vertically divided into thin strips, one thereof showing information for the left eye and the other showing information for the right eye, in alternation. Respective lens grids of plastic that supply the sub-images to the respectively correct eye are impressed in front of these strips. This method, for example, is employed in 3D postcards and has the great advantage that these stereograms need no further auxiliaries such as lenses or the like for viewing.

Holograms offer an especially advantageous possibility for a stereoscopic image carrier. These generate a spatial image impression by reconstructing the wave pattern impressed during registration, in a limited volume. A coherent light source (laser) is not compulsory for viewing, so that—as in the case of art holographs—illumination with an optimally punctiform white light source preferably ensue in the inventive utilization for a medical examination apparatus. This can be very easily realized by the aforementioned optical fiber illumination system, the light-guiding fibers terminating in a free end in front of the image carrier above the patient's head.

Among the advantages of a such a hologram for the inventive purposes is that the eyes of the viewer can focus on a virtual object that appears to lie far behind the plane of the film. A hologram therefore can also be mounted closely in front of the viewer's eyes without additional lenses, which is a pre-requisite given the intended purpose of optical enlargement of the examination space of a medical examination apparatus since, of course, the inside wall of the examination space is arranged at only a slight distance above the patient's face.

### DESCRIPTION OF THE DRAWINGS

FIG. 1 is a schematic view of the tubular examination space of a medical examination apparatus, particularly an MR scanner, with a stereoscopic image carrier that fills the field of vision of the patient arranged therein in accordance with the invention.

FIG. 2 is a cross-section through a modified image carrier with illumination from behind in accordance with the invention.

FIG. 3 is a view of the patient support table with a head coil outside the examination space in accordance with the invention.

FIG. 4 is a schematic cross-section through the examination space of a medical examination apparatus, with a stereoscopic image carrier is arranged within a head coil in accordance with the invention.

### DESCRIPTION OF THE PREFERRED EMBODIMENTS

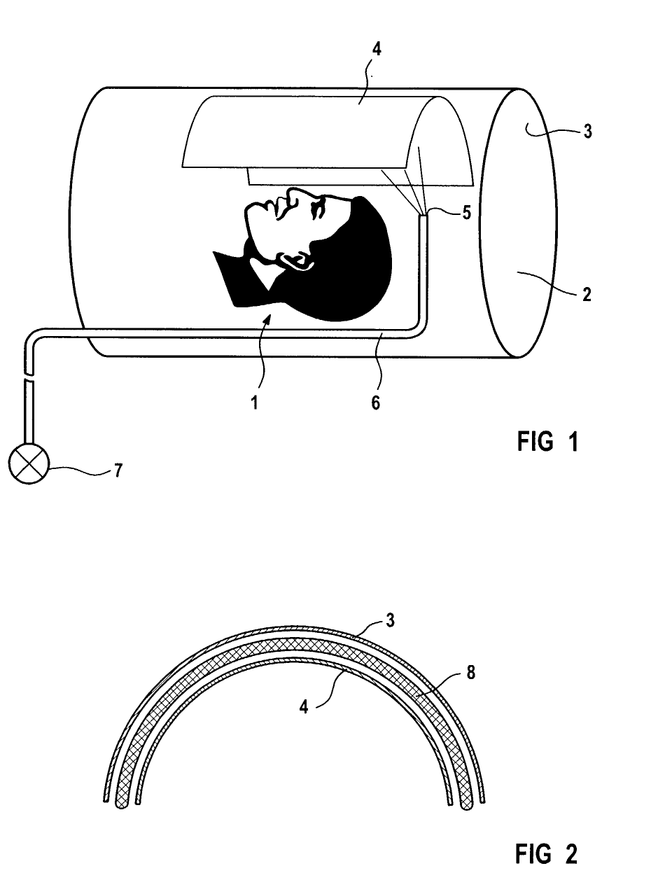

For simplicity, FIG. 1 shows only the head of a patient 1 within the tight, tubular examination space 2 of a medical examination apparatus, particularly a magnetic resonance scanner. In order to simulate a larger interior, a stereoscopic image carrier—when no head coil is employed—is arranged, preferably directly, at the inside wall 3 of the examination space over the patient's face. The image carrier, fashioned as a stereoscopic film, preferably is arced in the fashion of a cylindrical jacket in the illustrated exemplary embodiment. The image carrier 4 can be a lens grid image carrier or can carry a hologram. The illumination of the stereoscopic image carrier 4 in the exemplary embodiment of FIG. 1 ensues by means of a punctiform white light source that is formed by the light exit face 5 of a light guide 6 that terminates in a free end in front of the image carrier 4 above the head of the patient 1, and leads to a light source 7 arranged outside the examination space. In this way, the light source 7 cannot be disturbing given a magnetic resonance scanner, which is also true of the stereoscopic image carrier 4, that is preferably fashioned of plastic.

Instead of illumination of the stereoscopic image carrier 4 from the front, it can be illuminated from behind in a modified embodiment as shown in FIG. 2, namely by means of a diffusing lens 8 that can in turn be connected via a light guide with a light source arranged outside the examination space 2.

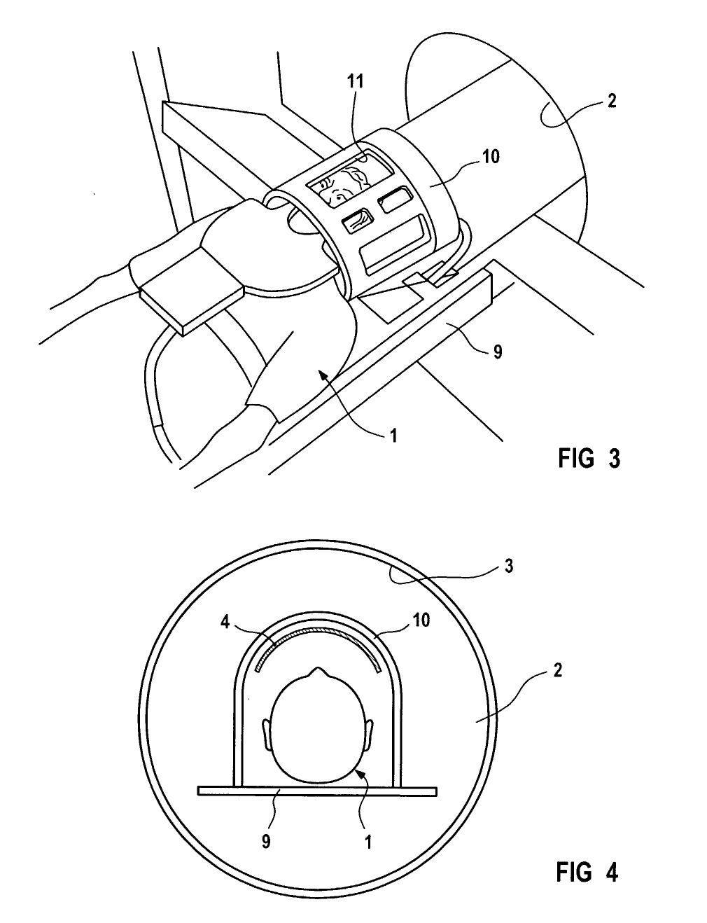

FIG. 3 shows the patient 1 on a patient support table 9 with which the patient 1 can be moved into the examination space 2, with a head coil 10 provided for the purpose of a magnetic resonance examination of the head. This affords the possibility of arranging the image carrier either above the cutout 11 on the head coil 10 or—as shown in FIG. 4—at the inside of the head coil 10. Alternatively, stereoscopic eyeglasses could be employed, these being either worn by the patient or being installed into the head coil 10.

Although modifications and changes may be suggested by those skilled in the art, it is the intention of the inventor to embody within the patent warranted hereon all changes and modifications as reasonably and properly come within the scope of his contribution to the art.

I claim as my invention:

1. A medical examination apparatus comprising:
a substantially tubular, tight examination space adapted to receive a patient therein, said patient having a field of view in said examination space; and
a stereoscopic image carrier, which optically simulates a space larger than said examination space, disposed in said examination space in said field of view.

2. A medical examination apparatus as claimed in claim 1 wherein said stereoscopic image carrier is free of metal.

3. A medical examination apparatus as claimed in claim 1 wherein said stereoscopic image carrier comprises two sub-images, and further comprising stereoscopic eyeglasses adapted to be worn by said patient for viewing a stereoscopic image produced by said two sub-images.

4. A medical examination apparatus as claimed in claim 3 wherein said stereoscopic image carrier is arced to form a portion of a cylindrical jacket.

5. A medical examination apparatus as claimed in claim 1 wherein said stereoscopic image carrier is a stereoscopic film.

6. A medical examination apparatus as claimed in claim 5 wherein said stereoscopic film is arced to form a portion of a cylindrical jacket.

7. A medical examination apparatus as claimed in claim 5 wherein said stereoscopic film includes a lens grid which distributes strip-shaped sub-images onto the eyes of said patient in alternation.

8. A medical examination apparatus as claimed in claim 5 wherein said stereoscopic film is a hologram.

9. A medical examination apparatus as claimed in claim 5 further comprising a light guide fiber for illuminating said stereoscopic film, said light guide fiber being in optical communication with a light source disposed outside of said examination space.

10. A medical examination apparatus as claimed in claim 9 wherein said light guide fiber terminates in a free end disposed in front of said stereoscopic film above a head of said patient.

11. A medical examination apparatus as claimed in claim 9 wherein said light guide fiber is disposed to illuminate said stereoscopic film from a rear of said stereoscopic film, and further comprising a diffusion lens disposed between said light guide fiber and said stereoscopic film.

12. A medical examination apparatus as claimed in claim 1 wherein said examination space has an interior wall, and wherein said image carrier comprises a stereoscopic film secured to said interior wall.

13. A medical examination apparatus as claimed in claim 1 further comprising a magnetic resonance head coil receivable in said examination space, and wherein said stereoscopic image carrier is a stereoscopic film disposed on an exterior of said magnetic resonance head coil.

14. A medical examination apparatus as claimed in claim 1 further comprising a magnetic resonance head coil receivable in said examination space, and wherein said stereoscopic image carrier is a stereoscopic film disposed on an interior of said magnetic resonance head coil.
